(19) United States
(12) Patent Application Publication
(10) Pub. No.: US 2007/0243263 A1
(43) Pub. Date: Oct. 18, 2007

(54) Antiviral Methods

(75) Inventor: Jeffrey A. Trogolo, Boston, MA (US)

Correspondence Address: EDWARD K. WELCH II;IP&L Solution, 4558 Ashton Court, Naples, FL, 34112, US

(73) Assignee: AgION Technologies, Inc.

(21) Appl. No.: 11/726,121

(22) Filed: Mar. 21, 2007

Related U.S. Application Data
(60) Provisional application No. 60/792,056, filed on Apr. 14, 2006.

(51) Int. Cl. A61K 33/38; A61K 33/06; A61K 33/42; A61K 47/48

(52) U.S. Cl. 424/604; 424/618

(57) ABSTRACT

Combinations of silver and copper ion sources or a single source of both silver and copper ions are found effective in methods for treating viral infections and for treating surfaces so as to eradicate viral contaminants and/or prevent subsequent contamination of said surfaces with viruses. These methods are particularly applicable in addressing SARS and avian flu viruses.

This application claims the benefit of U.S. Provisional Patent Application No. 60/792,056 filed on Apr. 14, 2006 entitled Antiviral Methods in the name of Jeffery A. Trogolo.

### FIELD OF THE INVENTION

The present invention relates to a method of treating surfaces for cleansing the same of viruses and/or for preventing the depositing and proliferation of viruses on surfaces. More specifically, the present invention is directed the treatment of various surfaces with certain inorganic antiviral compositions comprising a combination of silver ion and copper ion sources or a single source of both silver and copper ions.

### BACKGROUND OF THE INVENTION

The antimicrobial properties of a number of inorganic materials, especially metals such as silver, copper, zinc, mercury, tin, gold, lead, bismuth, cadmium, chromium and thallium, have long been known. Certain of these metals, especially silver, zinc, gold and copper, have enjoyed greater success due to their relatively low environmental and toxicological effects and high antimicrobial activity. More recently, antimicrobial agents that incorporate ionic forms of these metals, especially through an ion-exchange type mechanism, have achieved greater attention due to the higher bioactivity of the ionic versus the metallic form of these metals in various antimicrobial applications. Exemplary ion-exchange type antimicrobial agents include those wherein the ion-exchange carrier particles are ceramic particles including zeolites, hydroxy apatites, zirconium phosphates and the like. Antimicrobial agents based on zeolite carriers are disclosed in, for example, U.S. Pat. Nos. 4,911,898; 4,911,899; 4,938,955; 4,938,958; 4,906,464 and 4,775;585. Antimicrobial zirconium phosphates include those disclosed in, for example, U.S. Pat. Nos. 4,025,668; 4,059,679; 5,296,238; 5,441,717; and 5,405,644 and the Journal of Antibacterial Antifungal Agents Vol. 22, No. 10, pp. 595-601, 1994. Finally, antimicrobial hydroxyapatites powders include those disclosed in U.S. Pat. Nos. 5,009,898 and 5,268,174, among others.

Despite the relative, though restrained, commercial success of these ion-exchange type antimicrobial agents in attacking and preventing the growth or establishment of various microbial colonies on surfaces and the plethora of technical papers, articles, patents and the like describing these materials and their applications, very little if anything is said of their actual or potential efficacy in attacking viruses. Indeed, none of these commercial products are registered for or even suggest the possibility of antiviral applications. The few instances where viruses are mentioned are limited to inclusion within a list of other potential targets for their activity including, fungi, molds, bacteria and the like. None, however, demonstrate or make any definitive suggestion of their use in attacking viruses or of mentioning any specific viruses. This is consistent with the long held view of those skilled in the art that most antimicrobial agents, especially ionic agents, are largely ineffective against viruses. Instead, harsh measures, such as cleaning with bleach or other caustic agents, are needed to ensure good antiviral cleaning. While such actions can effectively cleanse a surface of viruses, they do not prevent the reoccurrence of viruses on said surfaces. Furthermore, these materials and methods oftentimes require special precautions against health and environmental effects and may adversely affect the surfaces being treated. In worst case scenarios, e.g., where a facility or plurality of surfaces in a given area are contaminated with pathogenic viruses, the whole of the affected area or surfaces are destroyed, often burned

While viruses have always been a concern to human and animal heath and well being, increased concern has arisen in the recent past with respect to a growing number of new, more virulent and pathologic viruses such as those associated with SARS and, more recently and of grave concern, avian flu viruses. While the former seems to have been contained, to some extent, a careful watch continues to guard against a reoccurrence of its outbreak. Of greater concern, however, is the recent and quickly expanding outbreak of avian flu virus. Though once thought to be contained to certain sections of Southeast Asia, avian flu virus has now been seen in Europe, Africa and all over the Asian continent. It is only a matter of time before it spreads world-wide. And, while essentially all outbreaks have been limited to birds, there have been growing reports of human infections. Indeed, dire warnings appear incessantly in the news and in print of a pending pandemic and the potential catastrophic consequences should the global expansion or outreach of the virus continue to accelerate and, more importantly, should the current form of the avian flu virus mutate so as to be more readily transmitted to humans and subsequently spread through human to human transmission.

While viruses have several methods of transmission from one host/victim to another, two key modes of transmission involve surfaces that act as a stopover for the viruses. In the first mode, a contaminated touch surface is touched by a human or animal whereby the human or animal is exposed to and infected by the virus. In the second mode, a surface involved with the flow of fluids or air is contaminated whereby the subsequent flow of the fluid or air mobilizes the virus and brings the virus into contact with a human or animal. Thus, one key response to any viral threat is to render susceptible surfaces antiviral or, if too late, to cleanse said surfaces of the virus and, preferably, concurrently render them antiviral from possible subsequent re-contamination.

Treatment of humans and animals infected with a virus depends upon the nature of the viral infection, its virulence and spread, and, in the case of animals, the nature of the animals at risk. Oftentimes, especially for non-virulent or non-pathogenic viruses, the common treatment is to make the patient, whether human or animal, comfortable, i.e., treat the symptoms, and let the virus run its course. This is because many, if not most, viruses are not responsive to traditional antibiotics or known medicaments. In humans, especially with the appearance of new viruses, isolation and/or hit or miss treatments are often the sole answer. As more and more becomes known of these viruses, agents are identified and/or developed which help attack the viruses or counter their effects. With domestic animals, the treatment methods oftentimes mimic those for humans; however, more often the animal is put to death. However, where there is concern for widespread contamination amongst animals, particularly those considered feedstock for the human population, and, perhaps, ultimate transmission to humans, particularly viruses like BSE and avian flu virus, the only action taken is the euthanasia of all infected and potentially infected animals. Oftentimes, this means the destruction of whole flocks, herds, etc. regardless of whether only one out of many tens, hundreds or even thousands of animals are infected, as well as destruction or decontamination of the areas in which the infected animals lived or passed though so as to prevent the subsequent contamination of other animals. While it is thought that the cost of such drastic action far outweighs the potential economic harm should the virus not be contained, such is of little consolation to the affected farmer/rancher/etc.

Thus, there exists a need for an effective method, especially a simple method, of cleansing a surface contaminated with a virus which is efficacious but does not require special precautions or equipment to accomplish. Indeed, there is especially a need for such a cleansing method that can be performed by the consumer without concern for adverse health and environmental effects.

Additionally, there exists a need for a method of providing an efficacious and long lasting treatment to surfaces which treatment manifests antiviral characteristics, killing viruses that come in contact with the surface and/or preventing the proliferation of viruses on the surface.

Finally, there exists a need for an effective treatment for those, especially those animals, infected with viruses, especially those associated with SARS and, more urgently, avian flu.

### SUMMARY OF THE INVENTION

In accordance with the teaching of the present invention there is provided a method of treating individuals and/or animals infected with a virus which method comprises treating the individual or animal, as a whole, or the infected area in the case of localized infections, with an antiviral composition comprising one or more sources of silver ions and copper ions. Specifically, the antiviral composition may comprise a silver ion source and a copper ion source or a single material may serve as the source of both the silver and copper ions. Though the source(s) may be ones wherein the ions are dissociated from a salt or an organometallic compound or, due to their extremely small particle size, colloidal or nano-sized particles of the metal itself. Preferably the source is one wherein the ions are generated by an ion-exchange mechanism whereby the silver and copper ions are released upon the exchange with other ions, especially naturally occurring ions. Most preferably there is provided a method of treating an infected individual or animal with a topical solution, ointment, lotion, transdermal functioning patch, hydrogel, or the like, or an oral or injectable solution containing an antiviral effective amount of an inert ion-exchange carrier having ion-exchanged copper and silver ions.

In accordance with a second aspect of the present invention, there is provided a method of treating surfaces contaminated with a virus for effectively eradicating said virus from said surface and, preferably, concurrently providing protection against the recontamination of, or at least the re-establishment of a virus on, the surface. Specifically, there is provided a method of treating infected surfaces with a solution or coating comprising one or more silver ion source(s) and one or more copper ions source(s) and/or a single source capable of providing both silver ions and copper ions. Most preferably there is provided a method of treating a contaminated surface with a solution, preferably a binder solution, or coating containing an antiviral effective amount of an inert ion-exchange carrier having ion-exchanged copper and silver ions.

In accordance with a third aspect of the present invention there are provided a plurality of methods for rendering surfaces, facilities, and articles of manufacture resistant to contamination by viruses or, if contamination occurs, resistant to further proliferation of those viruses. Specifically there is provided a method whereby a coating composition or other treatment comprising a one or more silver ion source(s) and one or more copper ions source(s) and/or a single source capable of providing both silver ions and copper ions is applied to the surfaces, facilities or articles of manufacture or any stock materials from which the foregoing are made. Alternatively, there is provided a method whereby a one or more silver ion source(s) and one or more copper ions source(s) and/or a single source capable of providing both silver ions and copper ions are incorporated into the composition of matter or stock materials from which the surfaces, facilities, or articles of manufacture or stock materials are made.

In its most preferred aspect, the present invention is applicable to the treatment, eradication and/or prevention of the proliferation of those viruses belonging to or derived from the family of the coronavirus, especially those associated with SARS, as well as those belonging to or derived from the avian flu virus.

### DETAILED DESCRIPTION OF THE DRAWINGS

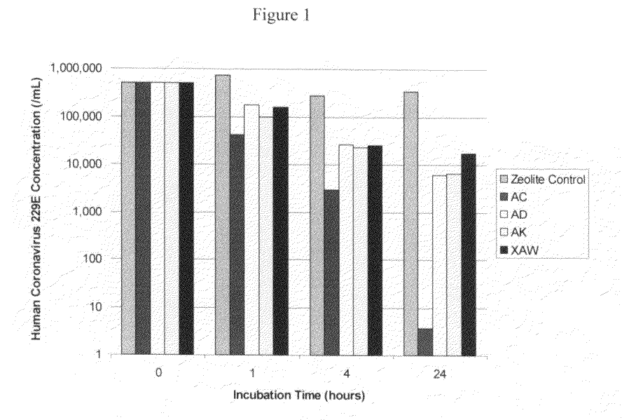

FIG. 1 is a logarithmic bar chart showing the efficacy of various silver ion sources, including those of the present invention, against the human coronavirus 229E.

### DETAILED DESCRIPTION OF THE INVENTION

All patent applications, patents, patent publications, and literature references cited in this specification, whether referenced as such, are hereby incorporated by reference in their entirety. In the case of inconsistencies, the present description, including definitions, is intended to control.

The present invention provides various methods for eradicating viruses from a surface and/or for rendering surfaces resistant to the proliferation of viruses. When used herein and in the appended claims, the terms “eradicate” or “eradication” mean that the antiviral methods employed in the practice of the present invention are capable of killing essentially all, e.g., 99%, preferably 99.9%, of the virus within 24 hours. Similarly, when used herein and in the appended claims, the terms “resistant” or “resistance” mean that the surfaces treated in accordance with the methods of the present invention will essentially prevent the proliferation of any viruses that may come in contact with said surfaces and, preferably, will kill all or essentially all viruses that may come in contact with said surfaces.

The antiviral agent according to the practice of the present invention comprises one or more materials that are capable of delivering a combination of silver and copper ions in an antiviral effective amount. By delivery we mean that the materials are able to release silver and copper ions, either through dissociation or an ion-exchange reaction, whereby they are free to be absorbed or adsorbed by or otherwise interact with the virus and/or its replication process. Suitable antiviral materials include copper and/or silver metallic and organometallic salts as well as silver and copper containing antibiotics, all having readily dissociable silver and/or copper atoms, especially in aqueous environments or mediums. Most preferably, the source of the silver and copper ions are ion-exchange materials having ion-exchange silver, copper or a combination of silver and copper ions, alone or in combination with another copper and/or silver ion source; provided that at least one source provides both silver and copper ions or at least one source provides at least one of silver or copper and a second source provides the other. Typically, the weight ratio of available copper to silver will be from about 1:20 to about 20:1, preferably from about 1:10 to about 10:1.

Suitable copper and silver salts are well known and include most any that have previously found utility in antibacterial and/or antifungal applications. Exemplary salts include the oxides, sulfides, chlorides, bromides, carbonates, nitrates, phosphates, dihydrogen phosphates, sulfates, oxalates, quinolinolates, acetates, benzoates, thiosulfates, phthalates, and the like of copper and silver. Specific examples include silver nitrate, silver oxide, silver acetate, cupric oxide, cuprous oxide, copper oxychloride, cupric acetate, copper quinolinolate, silver phthalate, and the like. Suitable silver and copper antibiotics include those previously sold under the following tradenames: Argenti, Acetas, Albargin, Argonin, Argyn, Argyrol, Largin, Lunosol, Novargan, Proganol, and Silvol. Other pharmaceutical or antibiotic silver materials include colloidal silver (especially that made by electrolysis or the electro-colloidal process), mild silver proteins (MSPs), silver sulfadiazine and nanocrystalline silver. Other pharmaceutical or antibiotic copper materials include copper water, copper sulfate, copper peptides, copper EDTA, copper PCA, and copper gluconate.

Another suitable silver and/or copper ion source is water soluble glasses that contain a silver and/or copper metal or salt. Suitable silver or copper containing water soluble glasses include those disclose in U.S. Pat. No. 5,470,585. By suitable adjustment of the glass composition, the dissolution rates in water can be controlled so as to control the release of the silver and/or copper ions.

The preferred silver and copper source(s) for use in the practice of the present invention are ion-exchange type ceramic particles having ion-exchanged copper and/or silver ions. Exemplary ion-exchange ceramic particles include, but are not limited to, aluminosilicates, zeolites, hydroxyapatite, and zirconium phosphates. Suitable hydroxyapatite particles containing silver and/or copper ions are described in, e.g., U.S. Pat. Nos. 5,009,898 and 5,268,174. Suitable zirconium phosphates are described in, e.g., U.S. Pat. Nos. 4,025,608; 4,059,679; 5,296,238; 5,441,717 and 5,405,644 as well as in the Journal of Antibacterial and Antifungal Agents, Vol. 22, No. 10, pp. 595-601, 1994. Finally suitable aluminosilicates and zeolites containing ion-exchanged silver and copper ions are described in, e.g., U.S. Pat. Nos. 4,911,898; 4,911,899; 4,938,955; 4,938,958; 4,906,464; and 4,775,585.

These ion-exchange antiviral agents are prepared by an ion-exchange reaction in which various cations present in the ceramic particles, for example, sodium ions, calcium ions, potassium ions and iron ions in the case of zeolites, are partially or wholly replaced with the antiviral copper and/or silver ions, preferably both. The weight of the antiviral metal ions, whether of one or both will be in the range of from about 0.1 to about 35 wt %, preferably from about 2 to 25 wt %, most preferably from about 4 to about 20 wt % of the ceramic particle based upon the total weight of antiviral metal containing ceramic particle. Where both the silver and copper ions are present in the same ceramic particle, each antimicrobial metal ion may be present in the range of from about 0.1 to about 25 wt %, preferably from about 0.3 to about 15 wt %, most preferably from about 2 to about 10 wt % of the ceramic particle based on 100% total weight of the ceramic particle and the weight ratio of silver to copper ions will generally be from 1:20 to 20:1, typically from 1:10 to 10:1, preferably from 5:1 to 1:5, most preferably from 2.5: to 1:2.5.

In addition to the copper and/or silver ions, the antiviral ceramic particles may also have other metal ions, such as zinc ions, which along with the silver and copper ions also provide antimicrobial characteristics. If present these additional antimicrobial metal ions will be present in the ranges set forth above for the silver and copper ions and will be included in the total weight of ion-exchanged metal ions also mentioned above.

In the preferred embodiments of the present invention the antiviral ceramic particles are zeolites, especially those of the type described in U.S. Pat. Nos. 4,911,898; 4,911,899 and 4,938,958. Suitable zeolites include natural and synthetic zeolites. “Zeolite” is an aluminosilicate having a three dimensional skeletal structure that is represented by the formula: XM_{2/n}O—Al₂O₃—YSiO₂-ZH₂O wherein M represents an ion-exchangeable ion, generally a monovalent or divalent metal ion; n represents the atomic valency of the (metal) ion; X and Y represent coefficients of metal oxide and silica, respectively; and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. Typically the surface area of these zeolites is at least 150 m²/g (anhydrous zeolite as standard) and the SiO₂/Al₂O₃ mole ratio is preferably less than 14 and more preferably less than 11. The ion-exchange capacities of these zeolites are as follows: A-type zeolite=7 meq/g; X-type zeolite=6.4 meq/g; Y-type zeolite=5 meq/g; T-type zeolite=3.4 meq/g; sodalite=11.5 meq/g; mordenite=2.6 meq/g; analcite=5 meq/g; clinoptilolite=2.6 meq/g; chabazite=5 meq/g; and erionite=3.8 meq/g. The present invention is not, however, limited to the foregoing zeolites.

In addition to the ion-exchanged antiviral metal ions within and on the exposed surface of the ion-exchange carrier particles, these carrier particles may also have some, albeit minor, amount of surface adsorbed silver and/or copper. These deposits on the exposed outer surfaces are often in the metal or metal salt form, especially oxides, and provide a quick, though comparatively short lived, release of the silver and/or copper ions upon exposure to water.

The antiviral ion-exchange materials, especially the zeolites, may also contain a discoloration agent, preferably one that is biocompatible and will not interfere with the antiviral performance of the silver and copper ions or other silver or copper ion sources, if present. Preferred discoloration agents include, but are not limited to, inorganic discoloration inhibitors such as ammonium. More preferably, the inorganic discoloration inhibitor is an ion-exchanged ammonium ion. The ammonium ions, if present, will be present in an amount of up to about 20 wt % of the ceramic particle though it is preferred to limit the content of ammonium ions to from about 0.1 to about 2.5 wt %, more preferably from about 0.25 to about 2.0 wt %, and most preferably from 0.5 to about 1.5 wt % of the ceramic particle.

A number of antimicrobial zeolites suitable for use in the practice of thee present invention are distributed by AgION Technologies, Inc., of Wakefield, Mass., USA, under the AgION trademark. One grade, AW10D, contains 0.6% by weight of silver ion-exchanged in Type A zeolite particles having a mean average diameter of about 3μ. Two additional grades, AG10N and LG10N, each contain about 2.5% by weight of silver ion-exchanged in Type A zeolite particles having a mean average diameter of about 3μ and 10μ, respectively. Another grade, AJ10D contains about 2.5% silver, about 14% by weight zinc, and between about 0.5% and 2.5% by weight ammonium ion-exchanged therein in Type A zeolite having a mean average diameter of about 3μ. Another grade, AK10D, contains about 5.0% by weight of silver ion-exchanged in Type A zeolite particles having a mean average diameter of about 3μ. However, the most preferred antimicrobial zeolite for use in the invention is that sold under the grade designation AC10D which consists of about 6.0% by weight of copper and about 3.5% by weight silver ion-exchanged in Type A zeolite particles having a mean average diameter of about 3μ.

The aforementioned silver and copper sources may be used in their neat form or in an encapsulated form wherein particles of the silver and/or copper ions source are individually encapsulated or, most preferably, a plurality of such silver and/or copper ion source particles are dispersed in individual microparticles of a hydrophilic polymer. The only limitation here is that the silver and/or copper ion source must be soluble and capable of water transport in and through the hydrophilic polymer or able to release the silver and/or copper ions within the hydrated hydrophilic polymer particle so that they may be transported in and through the hydrophilic polymer. The encapsulated form of the copper and silver ion sources provide a number of benefits including acting as concentrated reservoirs of the silver and copper ion source(s), providing for a controlled release of the silver and/or copper ions, and, depending upon the specific end use application, markedly increasing the amount of silver and copper ions capable of release for a given amount of silver and copper ion source.

Encapsulation of the silver and copper ion sources is especially beneficial with those silver and copper ion sources that, in use, are incorporated into polymer matrices, coatings and the like that are not hydrophilic and/or that do not allow the silver or copper ion source to migrate. This is because the antiviral activity is only seen if the silver and copper ions are able to come into contact with the virus. In these matrices, silver and copper ion sources that are not at the surface of the substrate where the virus is present or is able to be deposited, are ineffective and, thus, wasted. Those copper and silver ion sources that do migrate will do so and provide antiviral protection; however, since migration is constant, the antiviral activity tends to be short lived due to the constant depletion of the antiviral agent. On the other hand, encapsulation of the silver and copper ions sources markedly increases their effective size thereby increasing the likelihood that any portion of such particle may come in contact with a surface. And, since the silver and copper ions readily move in and through the hydrophilic polymer, all of the silver and copper ion source(s) within a given hydrophilic particle are available. Furthermore, because these hydrophilic polymers rely upon water transport, they only allow the release of the antiviral agent or silver and copper ions when conditions are appropriate, i.e., water and, in the case of the ion-exchange type agent, exchangeable cations are available. Finally, even when conditions are present for water transport, one can further control the rate of release or the antiviral agent by appropriate selection of the hydrophilic polymer, i.e., those with a lower degree of hydrophilicity will have a slower rate than those having a higher degree of hydrophilicity. Thus, these encapsulated copper and silver ions sources will have excellent controlled release and, thus, longevity and, depending upon the method of their use, higher overall antiviral performance for the given amount of silver and copper ions present.

Encapsulated silver and copper ion sources suitable for use in the practice of the present invention are disclosed in Trogolo et. al. (US2003-0118664 A1 and US2003-0118658, both of which are incorporated herein by reference). Though Trogolo et. al. primarily focused on encapsulating ion-exchange type agents, the teachings are equally applicable to most, if not all, of the other antiviral agents mentioned above. It is recognized, however, that certain agents may have limits on the process by which the encapsulation is accomplished, especially in the case of heat sensitive antibiotics and the like. Nevertheless, those skilled in the art will readily appreciate the application of the teaching of Trogolo et. al. to these other materials.

Generally speaking, the encapsulated silver and copper ion sources will comprise from about 5 wt % to about 75 wt %, preferably from about 10 to about 65 wt %, most preferably from about 20 wt % to about 50 wt % of the antiviral silver and/or copper source(s) based on the combined weight of the antiviral metal source(s) and the hydrophilic polymer. The encapsulated particles will generally have an average diameter of up to about 300μ, preferably from about 30μ to about 200μ, most preferably from about 50μ to about 150μ and an aspect ratio of from 1 to 4, preferable from 1 to about 2. Of course larger particles, e.g., up to 800μ, even up to 2000μ, and higher aspect ratios, e.g., up to 100, preferably less than 30, are possible, but not preferred, especially in coating applications or when to be taken or injected as a medicament. Similarly, though small, nano-sized particles are possible, it is preferred that the particles have an average diameter of 5μ or more, preferably 15μ or more. When speaking of average particle size, it is understood that a majority of the individual particles, preferably 75% or more, most preferably 90% or more, will fall within the designated range. In practice, the particles are most likely to be screened so as to ensure that substantially all particles fall within the desired particle size range.

Hydrophilic polymers suitable for use in making the encapsulated silver and copper ion sources are those that can absorb sufficient water to enable the encapsulated particle to exhibit good release of silver and copper ions. These polymers are characterized as having water absorption at equilibrium of at least about 2% by weight, preferably at least about 5% by weight, more preferably at least about 10% by weight, most preferably at least about 20% by weight, as measured by ASTM D570. Especially suitable and preferred hydrophilic polymers include those having water contents at equilibrium of from 50 to about 300% by weight, most preferably about 50 and to about 150% by weight.

The encapsulating hydrophilic polymers, hereinafter oftentimes referred to as the encapsulant, are typically comprised of substantial quantities of monomers having polar groups associated with them, such that the overall polymeric composition is rendered hydrophilic. The polar groups can be incorporated into the polymer main chain as in for example polyesters, polyurethanes, polyethers or polyamides. Optionally the polar groups can be pendant to the main chain as in for example, polyvinyl alcohol, polyacrylic acids or as in ionomers such as Surlyn®. Surlyn® is available from Dupont and is the random copolymer poly(ethylene-co-methacrylic acid) wherein some or all of the methacrylic acid units are neutralized with a suitable cation, commonly Na⁺ or Zn⁺². While not being limited by way of theory, it is believed that the inclusion of polar groups allows water to more readily permeate the polymer and consequently, to allow slow transport of the metal ion through the encapsulating polymer layer. Such encapsulants may be thermoplastic or they may be thermoset or cross-linked.

A number of specific hydrophilic polymers suitable for use as the encapsulant include, for example, (poly)hydroxyethyl methacrylate, (poly)hydroxypropyl methacrylate, (poly)glycerol methacrylate, and copolymers of hydroxyethyl methacrylate and/or methacrylic acid including styrene/methacrylic acid/hydroxyethyl methacrylate copolymers, styrene/methacrylic acid/hydroxypropyl methacrylate copolymers, methylmethacrylate/methacrylic acid copolymers, ethyl methacrylate/styrene/methacrylic acid copolymers and ethyl methacrylate/methyl methacrylate/styrene/methacrylic acid copolymers. Other suitable hydrophilic polymers and copolymers include polyacrylamide, hyaluronan, polysaccharides, polylactic acid, copolymers of lactic acid, (poly)vinyl pyrrolidone, copolymers of vinyl pyrrolidone, polyvinyl acetate, polyvinyl alcohol, and copolymers of polyvinyl alcohol and polyvinylacetate, polyvinylchloride, copolymers of polyvinylacetate and polyvinylchloride and hydroxyl-modified vinyl chloride/vinyl acetate copolymers, polyamides such as Nylon 6,6, Nylon 4,6 and Nylon 6,12, cellulosics and copolymers thereof, polyureas, polyurethanes and certain polyesters containing a high percentage (at least about 10% by weight, preferably at least about 25% by weight or more) of polyalkylene oxide. Preferred hydrophilic polymers and copolymers include polyhydroxyethyl methacrylate, polyacrylamide, polyvinylpyrrolidinone, polyurea, polysaccharides, polylactic acid, poly(meth)acrylic acid, polyurethane and copolymers thereof. Especially preferred hydrophilic polymers are the hydrophilic polyurethanes, such as the TECOPHILIC® polyurethane sold by Noveon (formerly Thermedics, Inc.) of Woburn, Mass. or a lightly cross-linked polymer based on n-vinylpyrrolidone and methylmethacrylate sold under the trade designation AEP Polymers by I H Polymeric Products Limited of Kent, England. Hydrophilic polyurethanes are those polyurethanes having a high ethylene oxide content, preferably as derived from polyethylene glycol, in the polymer chain. Typically, the ethylene oxide content is at least 40 percent by weight, preferably at least 50 percent by weight, based on the total polyol content.

The ultimate form of the composition comprising the antiviral copper and silver ion source(s) depends upon the specific application being contemplated. As mentioned above, there are several methods being contemplated by the present invention. The first involves the treatment of an infected individual or animal with a medicament comprising one or more source of silver and copper ions. The second involves a method of cleansing a surface of viral contamination comprising washing the surface, including the skin of an individual or animal, with a solution, soap, or other cleansing composition having incorporated therein one or more sources of silver and copper ions. The third involves a method of treating a surface, including the skin of an individual or animal, with a composition comprising one or more sources of silver and copper ions. Finally, the fourth method of the present invention comprises incorporating one or more sources of copper and silver ions into the manufacture of various substrates or the stock materials from which they made.

Treatment of infected individuals typically means the ingestion or injection of a medicament containing the copper and silver ion source(s). Ingestion may be by way or aqueous solutions or colloids containing the silver and copper ion source(s). Alternatively the silver or copper ion source could be incorporated into solid food, feedstock and the like. Injection may be by way of saline solutions containing the silver and copper ion source(s) or other known carriers for injectable antibiotics. Alternatively, the injection carrier may be a food grade oil which is injected subcutaneously to create a small pool of the medicament form which the silver and copper ions slowly release into the general anatomy of the infected individual or animal. Injectable medicaments may also be suitable for use of the encapsulated antiviral agent(s) since they will serve as additional reservoirs to further regulate the release of the silver and copper ions into the general anatomy of the individual or animal. Rather than having to engage in a regimen whereby a given dose of the medicament is consumed over an extended period of time, it is believed that a single injection of a medicament containing the encapsulated antiviral agent will suffice. Also, because of the regulated release from the encapsulant there is less concern with toxicity or other adverse consequences of silver and/or copper in the individual or animal as possible with intermittent injection or consumption of high doses of quickly released silver and copper ions.

Topical treatments may also be employed in the general treatment of a viral infection but are more likely to be employed in the case of viral infections on or in the dermis, eyes, etc. where the medicament may be directly applied to the site of the infection or injury. Otherwise, topical treatment is not likely to be employed unless the topical medicament takes the form of a suitable transdermal patch or the like, preferably one that includes a strong transdermal carrier material, such as DMSO. Furthermore, topical treatments, creams, lotions, and the like could be employed for prevention, especially for individuals who may, as a result of the nature of their work, e.g., medical personnel, laboratory personnel, research personnel, veterinary personnel, etc., come into contact with or have the possibility of coming into contact with viruses. The amount of the silver and copper ion source(s) to be incorporated into the treatment or medicament, or the amount of the treatment or medicament to apply, will vary depending upon the subject, the method of application, etc. Those skilled in the art may ascertain the same by simple experimentation in accordance with standard pharmaceutical practice in determining dosage. Preferably the amount should be such as will eradicate the virus over a period of ten (10) days or less.

The antiviral compositions of the present invention are also especially suited for use in treating various surfaces, especially touch surfaces and the like, that have been or may become contaminated with viruses. Compositions comprising the silver and copper ion source(s) suitable for use in cleaning contaminated surfaces include any cleaning solution, including tap water, provided that these solutions are free of chelating, sequestering or other agents that may bind the silver or copper ions, thereby preventing them from contacting and interacting with the viruses. Preferably, especially where the antiviral agent is one of the ion-exchange type agents, the cleaning solution will include one or more sodium, calcium or like cation containing salts, e.g., sodium bicarbonate, so as to facilitate or accelerate the ion-exchange process whereby the silver and copper ions are released. The cleaning solutions may also be in the form of hand soaps and body soaps that are used to cleanse an individual or animal that may have come or may have the potential for coming in contact with the virus through touch and/or airborne transmission, e.g., a poultry farmer in the case of avian flu virus. With all of these cleaner type antiviral compositions, it is important that the cleaning solution have a sufficient residence time on the substrate surface to act against any viruses. Most preferably, the cleaning solution will be left on the surface and allowed to dry in situ whereby a film of the antiviral copper and silver ion source(s), typically discontinuous, especially in the case of the particle type source(s), will be left of the substrate surface. Typically, these cleaning solutions will comprise from about 0.1 to about 30 wt %, preferably from about 0.5 to 20 wt %, of the silver and copper ion source(s) based on the total weight of the cleaning formulation. Higher or lower concentrations are possible: the former being especially desirable where fast action is needed.

While the foregoing cleaning solutions effectively eradicate the viruses from the surface of the substrate being cleaned, the effectiveness of the antiviral activity is not long lived since any subsequent wiping, rinsing, etc., of the surface will remove substantially all, if not all, of the residual silver and copper ion sources. Thus, for providing immediate as well as long-term antiviral protection to a surface or substrate, it is preferred to treat the surface of the substrate with a coating that contains the silver and copper ion source(s). Most any known coating composition may be employed in the practice of the present invention provided that they are free of any chelating, sequestering or other agents that may bind the silver or copper ions. While the preferred coatings will have the silver and copper ion source(s) incorporated directly into the coating composition prior to its application to the intended substrate, an alternative method involves the application of the base coating composition to the substrate followed by the application, typically by dusting, of the silver and copper ion source(s) to the wetted surface of the coated substrate before the coating sets or cures.

Coatings are typically of two types, those comprising or containing a binder, most typically a resin or polymer, either in solution or suspended in a liquid carrier (e.g., a dispersion, colloid or emulsion), which forms a film upon evaporation or loss of the solvent or carrier, as appropriate, and those which are free or substantially free of solvents or carriers and involve at least one physical transformation of the coating material as applied to the substrate, either from a liquid or flowable 100% solids material to a solid or semi-solid film or layer of a polymer material (i.e., curable coatings) or from a particulate solid material to a substantially uniform film or layer of the solid material through heat (powder coatings). The curable coatings are perhaps the most diverse and may take a number of forms in and of themselves. For example, they may comprise one-part systems that cure or set upon exposure to certain environmental conditions, e.g., heat, light, moisture. Alternatively, they may comprise two- or more-part systems that are essentially shelf stable as long as the parts remain isolated from one another but cure or become curable upon mixing of the two or more parts, e.g., coatings that contain a catalyst in one part and an initiator in another.

Further, the coatings of the present invention may be single layer or multi-layered systems wherein each layer may have originated from a single or multi-part coating composition and provides different physical properties and/or antiviral benefits. A preferred multilayered coating system is one wherein a hydrophilic coating is applied as a topcoat over a non-hydrophilic coating. These systems provide excellent short term or immediate antiviral activity as well as long term durability and antiviral activity and are disclosed in, e.g., Trogolo et. al. US 2005/0287375, which is incorporated herein by reference. Selection of the coating both in terms of its composition, its form and, if appropriate, cure modality, will depend upon the specific substrate to be treated, the method of application, and the environmental and use conditions to which it will be exposed and, in following, the physical properties desired of the coating material itself. Since conventional coatings may be modified for use in the practice of the present invention, those skilled in the art will select the appropriate coating for their given application.

Generally speaking, the chemistry or formulation of the coating compositions vary widely and, as noted above, are selected based on the desired physical properties of the coating compositions, the mode of application (e.g., solution based, curable 100% solids, powder coating, etc.), the pot life (if applicable) and the environmental conditions to which they are exposed in use. Typically, in the case of thermoset coatings the choice of polymer or polymerizable components is based on the cure method and pot life as well as the adhesion, wear, and appearance characteristics or properties. In the case of thermoplastic coatings, selection of the thermoplastic polymer is based on the solvent needed and/or the ease of application, especially as powder coatings, as well as their adhesion, wear and appearance characteristics or properties. For high wear or stress environments or applications, it is preferred that the coatings be non-hydrophilic. However, for other applications, especially where it is desired to have a coating of a defined life as in the case of the top coat of a multilayered coating system, especially an erosive coating system, it is preferred that the coating be a hydrophilic coating.

Suitable thermoplastic polymers include, but are not limited to, polypropylene, polyethylene, polystyrene, ABS, SAN, polybutylene terephthalate, polyethylene terephthalate, nylon 6, nylon 6,6, nylon 4,6, nylon 12, polyvinylchloride, polyurethanes, silicone polymers, polycarbonates, polyphenylene ethers, polyamides, polyethylene vinyl acetate, polyethylene ethyl acrylate, polylactic acid, polysaccharides, polytetrafluoroethylene, polyimides, polysulfones, and a variety of other thermoplastic polymers and copolymers. Suitable thermoset or cross-linkable coatings include, but are not limited to, phenolic resins, urea resins, epoxy resins, including epoxy-novalak resins, polyesters, epoxy polyesters, acrylics, acrylic and methacrylic esters, polyurethanes, acrylic or urethane fortified waxes and a variety of other thermoset or thermosettable polymers and copolymers. Especially preferred thermoset coating systems are those based on epoxy resins, whether 100% solids or aqueous dispersions/latexes, due to their excellent adhesion to a variety of substrates and durability. Suitable epoxy resin systems include those sold by Corro-Shield of Rosemont, Ill. as well as Burke Industrial Coatings of Vancouver, Wash.

Hydrophilic polymer coatings include coatings comprising any of the aforementioned hydrophilic polymers used in making the encapsulated antiviral agents, as discussed above. Alternatively, coatings of certain traditional non-hydrophilic polymers may be made hydrophilic by blending a hydrophilic polymer with a non-hydrophilic polymer and/or cross-linkable coating polymer precursor. A preferred blend is made by using a supporting polymer comprising a plurality of functional moieties capable of undergoing crosslinking reactions, said supporting polymer being soluble in or emulsified in an aqueous based medium; and a hydrophilic polymer, said hydrophilic polymer being associated with the supporting polymer as described in U.S. Pat. No. 6,238,799. The ratio of the hydrophilic to non-hydrophilic and/or cross-linkable polymer depends on the hydrophilicity of the hydrophilic polymer and the desired hydrophilicity of the resultant blend.

As noted previously, coatings produced in accordance with the teaching of the present invention may comprise a single layer or two or more layers, each of which incorporates the copper and silver ion source(s). Single layer coatings are preferred due to their simplicity of application; however, as noted above, most coating applications do not allow for the use of hydrophilic polymers and, therefore, there is concern for the silver and copper ion source(s) contained within the coating and below the surface thereof. This concern may only be temporary in the case of coated surfaces that are subject to wear during use, especially floors. Alternatively, even those coatings, as well as all non-hydrophilic coatings where skinning over is a concern, can be activated by quickly eroding the surface layer of polymer coating. Depending upon the physical properties of the coatings, such may be achieved simply by buffing and/or lightly sanding the surface. Yet another alternative would be to employ hydrophilic polymer encapsulated copper and silver ion source(s) as discussed above.

In accordance with the practice of the present invention, the coating, or either or both the top coat and the base coat in the case of multilayered coatings, will generally contain from about 1 to about 30%, preferably from about 5 to about 20% and most preferably from about 5 to about 10%, by weight of the copper and silver ion source(s) based on the total weight of film forming materials. The foregoing ranges also hold true for those coatings where encapsulated copper and silver ion source(s) are employed except that the weight percent of the copper and silver ion source(s) is based on the weight of just the antimicrobial agent exclusive of the encapsulant material.

For those coating compositions wherein the copper and/or silver ion source is an ion-exchange type antiviral agent, the coating may also include a dopant for enhancing the initial release, and hence activity, of the copper and silver ions. Specifically, dopants provide a ready source of cations that exchange with and replace the silver and copper metal ions in the ion-exchange ceramic particles, thereby facilitating release and transport of these ions. Preferred dopants include, but are not limited to inorganic salts of sodium such as sodium nitrate.

Finally, the coating formulations, especially the top coat formulation in the case of multi-layered coating systems, may also contain other additives such as UV or thermal stabilizers, adhesion promoters, dyes or pigments, leveling agents, fillers and solvents. The specific additives to be use and the amount by which they can be used in the coating formulations of the present invention will depend upon the end use application and the choice of the polymer. Generally speaking, though, the selection and level of incorporation will be consistent with the directions of their manufacturers and/or known to those skilled in the art.

Coating compositions comprising the silver and copper ion source(s) may be made in accordance with any conventional method for coating preparation. Generally, the copper and silver ion source(s) is mixed with the coating formulation during or immediately following its preparation or as a separate additive to the fully formulated coating prior to shipment and/or application. The latter is especially preferred where there is any concern that the antiviral additive may adversely interact with the components of the coating composition during production and/or long-term storage. In the case of powder coatings, the silver and copper ion source(s) may be blended with the preformed powder coating particles or they may be incorporated into the pre-mix for the same, thereby dispersing the antimicrobial agent into the powder coating particles themselves.

Similarly, the coating compositions are applied by any of the methods known in the art, including spraying, brushing, rolling, printing, dipping and mold coating, powder coating, etc. The selection and thickness of the coating or coatings, in the case of multi-layered systems, can vary widely and depends upon the application requirements and limitations. For example, a high wear environment may require at thicker coating, especially one of good durability and/or wear resistance. The thickness of the coating, or the base coat in the case of multi-layered coatings, may also be a function of life of the substrate to which it is applied or, if the coating is periodically refinished or removed and replaced, the intended life of the coating itself. Generally, the thickness is the same as would be used for such coating compositions in the absence of the copper and silver ion source(s). Since, in practice, the copper and silver ion source(s) may be added to commercially available coating compositions, typically the thickness and rate of application will be as recommended by the manufacturer of the same. However, given the aforementioned issues with copper and silver ion source(s) that lie below the surface of non-hydrophilic coating or are not mobile within the coatings, the additional factors come into consideration as discussed below.

When the top coat polymer is a non-hydrophilic composition, especially a skin forming non-hydrophilic composition, it is especially preferred that the thickness of the cured top coat is, at most, slightly thicker than, but preferably the same as or less than, the average particle size or, in the case of encapsulated antiviral agents, the effective particle size of the antiviral agent and/or that a higher loading of the antiviral agent is employed so as to increase the amount of antiviral agent at or near the surface. Average particle sizes of slightly less than the thickness of the coating are possible since the distribution of particles will still provide a good number of particles in excess of the coating thickness and the coating thickness itself oftentimes varies across the surface of the substrate to which it is applied. Thus, the goal is to ensure that an adequate number of antiviral particles have not skinned over so that a sufficient level of silver and copper ion release is capable without having to wear away or remove the skin. In this respect one would want for at least about 30%, preferably at least about 40%, of the antiviral particles to have a diameter of equal to or less than the thickness of the coating. Though one could add greater quantities of antiviral agents whose average particle size is more than a micron or so less than the thickness of the coating, such would not be economical, especially in relatively low cost applications.

Preferred coatings for use in the practice of the present invention, whether as the sole coat or as a base or topcoat, will be such that the particles of the antiviral agent do not readily settle in the coating formulation once applied. Settling has the same effect as skinning as the coating material flows over the top of the particles as they settle in the composition. Thus, coatings having a high viscosity, e.g., typical of house paint or higher, or manifesting thixotropic behavior are especially preferred. In essence, it is especially desirable that the viscosity of the coating composition be such that, following application, the coating composition cures before any significant settling has occurred, particularly where the thickness of the coating as applied to the substrate is to be greater than the particle size of the antimicrobial agent. Another way of achieving such high concentrations of antiviral agent at the surface is the dusting of the wet, uncured, coating material with the copper and silver ion source(s) following the application of the coating to the substrate surface but before cure of the same, as mentioned earlier.

The versatility and ease of use of coating compositions comprising the silver and copper ion source(s) make them especially desirable, especially with respect to their ability to retroactively treat and render antiviral surfaces already in use. They may be applied to any of a number of surfaces or articles of manufacture, regardless of their manufacture, i.e., whether they are composed of metal, plastic, wood, glass, etc., with the selection of the specific coating matrix being dependent in part upon the surface to be coated and the conditions to which it is exposed so as to ensure sufficient surface wetting and adhesion. Such characteristics are known in the art and supplied by manufacturers of various coating materials. Suitable applications for the coatings of the present invention include, but are not limited to, building and work surfaces including walls, floors, ceilings, doors, counter tops, etc.; touch surfaces such as light switches, telephones, cutting boards, shelving, door and drawer handles and knobs, etc.; air and fluid flow surfaces such as ventilation conduits, ducts, air filters, water spigots, water taps, water filters, etc.; as well as various articles of manufacture including mats, containers, conveyor belts, appliances, and the like. Other surfaces include chemical storage tanks, animal feed dispensers and bins, water troughs, cooling water systems and pipes, air conditioning systems, and the like. In particular, the coating systems of the present invention are especially suited for use in animal husbandry, processing and rendering facilities; food preparation and processing facilities; pharmaceutical and biotechnology related manufacturing, testing and processing facilities; and in transport vehicles and storage facilities/apparatus associated therewith including, for example, the inner walls of grain silos, rail cars, tanker trucks, bulk storage containers, pens, hen houses, etc. as well as other structures and articles of manufactured associated and/or employed therewith

Finally, another way in which various articles of manufacture and substrates may be rendered antiviral is by the incorporation of the silver and copper ion source(s) directly into the matrix of the materials from which they are made. Specifically, the copper and silver ion source(s) may be directly compounded into various resins and polymer compositions, especially thermoplastic compositions, which are subsequently molded, extruded, pultruded, etc. into a finished good or a stock material used in making a finished good or substrate. Similarly, they may be incorporated into the precursor materials for various composite and thermoset compositions concurrent with or prior to their molding or forming process to make finished goods or stock materials. However, since the vast majority of thermoplastics are not hydrophilic and, in any event, hydrophilic materials have very limited applications, various specialized plastic forming and processing methods may be employed in order to minimize that portion of the silver and copper ion source(s) that are not accessible and, thus, ineffective until exposed. For example, films, sheet and articles of manufacture may be made by co-extrusion methods whereby the outer exposed surface(s) carries the silver and copper ion source(s) while the inner or center layers or a surface where antiviral activity is not needed, is free of the silver and copper ion source(s). Other methods include over-molding, rotational molding, and the like where only the exposed polymer material contains the copper and silver ion source(s). Similarly, one may prepare laminate structures where the exposed laminate surface incorporates the silver and copper ion source(s) but the under layers or substrate to which they are applied or adhered do not.

As noted above, the copper and silver ion source(s) may be incorporated into most any plastic or polymer material, whether thermoplastic or thermoset, including silicones and the like. Exemplary thermoplastics into which the silver and copper ion source(s) may be incorporated include, but are certainly not limited to, any of those mentioned previously with respect to thermoplastic coating materials, including, or as well as, polyesters, polyolefins, polyetheresters, polyetherimides, polyimides, polyamides, polyphenylene ethers, polystyrenes, ABS, polycarbonates, thermoplastic elastomers (TPEs), polyvinylchloride, polyvinylethers, polyvinylacetates, polyacrylates and poly(meth)acrylates, and the like. Exemplary thermoset materials include, but are not limited to those mentioned previously with respect to the thermoset coating materials including, or as well as, thermosetting polyesters, epoxy resins, thermosetting polyurethanes, alkyds, phenol-formaldehyde resins, urea-formaldehyde resins and the like.

The silver and copper ion source(s) is incorporated into the polymer material by any known method suitable for the given silver and copper ion source(s) and the selected polymer materials. For example, melt blending and solution blending are especially suited for thermoplastic materials, the latter especially where the silver and copper ion source(s) may be heat sensitive at or near the melt temperatures of the polymer. Otherwise, especially for thermoset materials, the silver and copper ion source(s) is incorporated into one or more of the prepolymers or other materials used in forming the polymer materials prior to polymerization thereof.

The amount of silver and copper ion source(s) incorporated into the polymer materials is typically from about 0.1 to about 30 wt %, preferably from about 0.5 to 20 wt %, based on the total weight of the polymer composition. As with the coatings, there is little by way of limitation as to the end-use applications to which thermoplastic and thermoset materials incorporating the silver and copper ion source(s) may be applied. However, the use of these modified plastic materials is especially desirable for those applications and/or articles of manufacture that are subject to considerable wear and erosion in use. For example, conduits, door handles, feeding bins, etc. where a coating, even a thick coating is likely to wear off before the end of the useful life of the article itself.

As discussed above, the combination of copper and silver ions provides effective action against viruses generally and, in particular, those viruses associated with or similar/linked to those viruses associated with SARS and avian flu. Thus, especially with respect to the SARS virus, the use of the copper and silver ion source(s) is especially relevant in articles of manufacture and in coatings applied to articles of manufacture, substrates and surfaces were common touching is associated or pathways exist for contaminating a large number of people from a single source. Thus, food processing and preparation areas and utensils; food service and related areas such as sinks, dish and glass washers, and the like; community/public baths and bathrooms; mass transit vehicles including trains, subways, aircraft, buses and the like; health care facilities like hospitals, emergency centers, health clinics and the like; will be especially benefited from the present invention. For viruses linked more closely to animals, at least at inception, such as avian flu virus, applications in animal husbandry such as the treatment of pens, cages, feed stores, feeding bins and tanks, water troughs, fences, barns, animal transport vehicles, slaughter houses and the like will be especially beneficial. Additionally, the treatment of public areas where the infected animals are likely to congregate, such as fountains, bird baths, feeders and the like, in the case of birds, may also help deter the spread of avian flu virus.

The following examples are presented as demonstrating the unexpected synergy of the combination of copper and silver ion sources in eradicating and preventing the spread of viruses, especially viruses related to the SARS coronavirus and human nonovirus, known human pathogens. These examples are merely illustrative of the invention and are not to be deemed limiting thereof. Those skilled in the art will recognize many variations that are within the spirit of the invention and scope of the claims.

### EXAMPLE 1

A first set of viral testing was conducted on the human coronavirus strain 229E and the feline infectious peritonitis virus (FIPV), both obtained from American Type Culture Collection of Rockville, Md. (ATCC #VR-740 and ATCC #VR-990, respectively). These viruses are often used as surrogates for SARS coronavirus (ScoV). In this set of experiments, flasks containing suspensions of five different zeolite materials were inoculated with the aforementioned viruses, the original titer being 5.0×10⁵ TCID₅₀/ml for the human coronavirus and 5.6×10³ TCID₅₀/ml for the FIPV virus. All five zeolite materials were type A zeolites, the first, Zeolite A, was unmodified. Four modified zeolites were prepared by ion-exchange to incorporate various metal ions as follows; Zeolite B—3.5 wt % silver and 6.5 wt % copper, Zeolite C—20 wt % silver, Zeolite D—5.0 wt % silver and 14% zinc, and Zeolite E—a combination of 80% zinc oxide and 20% zeolite having 0.6 wt % silver and 14 wt % zinc. All zeolites were obtained from AgION Technologies, Inc. of Wakefield, Mass. The suspensions comprised 30.0 ml of a 0.01 mol/liter phosphate buffered saline (PBS, pH 7.4 from Sigma-Aldrich, St. Louis, Mo.) with 10 mg of the suspended zeolite. A control without any antiviral agent was also included. The flasks were placed on an orbital shaker (200 rpm) at room temperature (23° C.) and sampled at 1, 4 and 24 hours using the Reed-Muench titration method to determine the TCID₅₀ (tissue culture infectious dose that affected 50% of the cultures). Each experiment was conducted in duplicate. Table 1 sets forth the results. All results are presented as the mean of the duplicate tests with the specific values reported as TCID₅₀ counts per milliliter (ml). FIG. 1 is a logarithmic plot of the test results with the coronavirus 229E for ease of review.

The results shown in Table 1 and FIG. 1 demonstrate the marked improvement of the silver/copper zeolite (Zeolite B) as compared to the highly loaded silver zeolite (Zeolite C) or the combination of silver zeolite and zinc oxide (Zeolite D). Although a minor effect was noted with Zeolites C and D against the human coronavirus 229E, such was marginal at best. Only the silver/copper zeolite (Zeolite B) showed significant and efficacious results against both viruses tested.

TABLE 1

| | Time | | Zeolite | | | | |
|---|---|---|---|---|---|---|---|
| Virus | (hours) | Control | A | B | C | D | E |
| 229E | 1 | 1.0E6 | 7.3E5 | 4.2E4 | 1.9E5 | 1.0E5 | 1.6E5 |
| | 4 | 1.0E5 | 2.8E5 | 2.9E3 | 2.7E4 | 2.3E4 | 2.5E4 |
| | 24 | 1.3E5 | 3.5E5 | <3.7* | 6.1E3 | 6.6E3 | 1.8E4 |
| FIPV | 1 | 4.6E3 | 5.6E3 | nd | 7.2E3 | | 3.2E3 |
| | 4 | 7.2E3 | 6.1E3 | <3.7* | 4.0E3 | | 3.5E3 |
| | 24 | 6.8E3 | 4.0E3 | <3.7* | 5.6E3 | | 3.2E3 |

nd—not determined
*detection limit

### EXAMPLE 2

A second set of viral testing was conducted on the human coronavirus strain 229E and the feline calicivirus strain F-9, both obtained from American Type Culture Collection of Rockville, Md. (ATCC #VR-990 and ATCC #VR-782, respectively). Feline calicivirus, an accepted surrogate for the human NoV pathogenic virus, is an enveloped virus, a form of virus that is typically more resistant to environmental conditions and the action of antimicrobial/antibiotic agents. In this set of experiments, Zeolite B from Example 1 was compounded into polyethylene, at two different loadings, 5 wt % and 10 wt %, and coupons molded from the compounded materials. Each polyethylene coupon was inoculated using a sterile glass rod with 0.1 ml of diluted virus: the original titer of each virus being 4.05×10⁵ TCID₅₀ for human coronavirus and 5.0×10⁶ PFU for feline calicivirus. The coupons were placed in humidity chambers (˜95% relative humidity) at room temperature (23° C.). Each coupon was sampled using a sterile polyester swab and dipped in 1.0 ml of D/E neutralizing broth (obtained from Remel of Lenexa, Kans.) immediately following inoculation and at 1, 4 and 24 hours following inoculation for titer determination. Each experiment was conducted in triplicate. The titers were determined using the Bidawid plaque-forming assay for the feline calicivirus and the aforementioned Reed-Muench TCID₅₀ method for the conronavirus.

The results of these evaluations are shown in Table 2. As shown, the silver/copper zeolite modified polyethylene, despite the fact that this polymer is non-hydrophilic, provided a marked reduction in the number of viruses after 24 hours. The difference in the results between the suspensions of Example 1 and the polymer coupons of this Example 2 is indicative of the fact that non-migrating silver and copper ion sources, especially ion-exchange type sources, that are not at the surface of the polymer article are not available to provide silver and copper

TABLE 2

Zeolite B
Concentration

| Virus | Time (hours) | Control^{†} | 5 wt % | 10 wt % |
|---|---|---|---|---|
| 229E | 1 | 3.4E5 | 47E4 | 6.3E4 |
| | 4 | 2.5E5 | 1.2E5 | 1.5E4 |
| | 24 | 1.7E5 | 5.8E3 | 6.8E3 |
| F-9 | 1 | 4.6E6 | 2.8E6 | 1.1E6 |
| | 4 | 3.4E6 | 1.1E6 | 5.4E5 |
| | 24 | 2.0E6 | 4.8E2* | 1.5E3 |

^{†}plastic coupon contained no zeolite
*the mean of two tests, the third was discarded as clearly anomalous.

ions. As enumerated above, increasing the amount of the zeolite at the surface and/or moderate abrasion of the surface of the coupons, such as with a fine sandpaper, would increase the number of exposed zeolite particles, thereby increasing their efficacy. Nevertheless, it is clear that the combination of silver and copper ions is efficacious against the viruses tested.

### EXAMPLE 3

A further set of experiments was conducted on the H5N1 bird influenza virus obtained from the Ministry of Agriculture in China using two different solutions, one containing a silver/copper zeolite, Zeolite B from Example 1, and the other containing another type A zeolite, Zeolite F, containing 3.5 wt % silver and 14 wt % zinc. In this experiment, 10-day old SPF chick embryos obtained from the China Agricultural Scientific Academy were inoculated with a solution that contained both the H5N1 virus and four different concentrations (10, 20, 100 and 200 mg/ml) of each of the two different zeolites in sterilized normal saline. Initially, two control studies were performed, one inoculating embryos with a series of 0.1 ml solutions of each of different concentrations of the zeolite solutions and the other inoculating the embryos with 0.1 ml solutions of a 10-times series dilution of the H5N1 virus. According to the first control study, none of the zeolite solutions were found to cause any visual pathologic change to the chick embryos. According to the second control study, the EID₅₀ of the H5N1 virus in the SPF chick embryos was found to be 10^{7.5}.

The inoculums for performing the tests of this series of experiments was prepared in accordance with the Klein-Defors suspension method and contained a titer 10^{7.5} H5N1 virus together with the designated concentration of the zeolite. Each inoculum was then allowed to stand for 10 minutes at 20±1° C. following which each was then subjected to a 10-times series dilution with sterilized normal saline. Five chick embryos were then injected with 0.1 ml of each dilution of each inoculum. The inoculated embryos were then placed in an incubator (37° C.) for 96 hours. Following that time, the allantoic fluid was removed from each embryo and tested using the hemagglutination (HA) test: a positive HA test being indicative of infestation of the chick embryo.

The results of this study are presented in Table 3. As indicated the silver/copper zeolites performed markedly better than the silver zeolites and produced results that indicate the viability of this combination as a means of treating and preventing the spread of bird influenza virus.

TABLE 3

Zeolite Concentration (mg/ml)

| Zeolite | 10 | 20 | 100 | 200 |
|---|---|---|---|---|
| Zeolite B | 0 | 0 | 99 | 100 |
| Zeolite F | 0 | 0 | 90 | 99.9 |

Although the present invention has been described with respect to the foregoing specific embodiments and examples, it should be appreciated that other embodiments utilizing the concept of the present invention are possible without departing from the scope of the invention. The present invention is defined by the claimed elements and any and all modifications, variations, or equivalents that fall within the spirit and scope of the underlying principles.

We claim:

1. A method of treating individuals or animals or both infected with a virus which method comprises treating the individual or animal, as a whole, or the infected area in the case of localized infections, with an antiviral composition comprising one or more sources of silver ions and copper ions, said one or more sources of silver ions and copper ions being capable of releasing said silver and copper ions in an antivirally effective amount.

2. A method of treating surfaces contaminated with a virus for effectively eradicating said virus from said surface, said method comprising the step of cleaning the surface with a cleansing solution containing one or more sources of silver and copper ions, said one or more sources of silver ions and copper ions being capable of releasing said silver and copper ions in an antivirally effective amount.

3. A method of treating surfaces for preventing the contamination of said surface with a virus, said method comprising the step of applying to said surface a coating comprising one or more sources of silver and copper ions, said one or more sources of silver ions and copper ions being capable of releasing said silver and copper ions in an antivirally effective amount.

4. A method of producing articles of manufacture and stock materials for use in the production of articles of manufacture which are resistant to contamination with viruses, which method comprises manufacturing said articles of manufacture and stock materials from materials which have incorporated therein one or more sources of silver and copper ions, said one or more sources of silver ions and copper ions being capable of releasing said silver and copper ions in an antivirally effective amount.

5. The methods of any of claims 1, 2, 3 or 4 wherein the one or more sources of silver and copper ions comprises at least two sources, at least one of which is a source of silver ions and at least of which is a source of copper ions.

6. The methods of any of claims 1, 2, 3 or 4 wherein the one or more sources of silver and copper ions comprise a single source which serves as a source of both silver and copper ions.

7. The methods of any of claims 1, 2, 3 or 4 wherein the one or more sources of silver and copper ions comprises two sources, one of which serves as a source of both silver and copper ions and the other as an additional source of one of silver or copper ions.

8. The methods of any of claims 1, 2, 3, or 4 wherein the weight ratio of copper to silver is from 20:1 to 1:20.

9. The methods of any of claims 1, 2, 3, or 4 wherein the weight ratio of copper to silver is from 10:1 to 1:10.

10. The methods of any of claims 1, 2, 3, or 4 wherein the source(s) of the copper ions, the silver ions or both is an ion-exchange type ceramic carrier having ion-exchanged silver and copper ions.

11. The method of claim 10 wherein the ion-exchange type ceramic carrier is selected from the group consisting of hydroxy apatites, zirconium phosphates, aluminosilicates, and zeolites.

12. The method of claim 10 wherein the ion-exchange type ceramic carrier is a zeolite.

13. The method of claim 10 wherein the source(s) further includes a copper ion or silver ion source which is not an ion-exchange type ceramic carrier.

14. The method of any of claims 1, 2, 3, or 4 wherein the virus is a coronavirus, the SARS virus or the avian flu virus or a mutation of any one of the foregoing.

15. A medicament for the treatment of viruses comprising a carrier and an antivirally effective amount of one or more sources of silver ions and copper ions.

16. The medicament of claim 15 which is administered orally or by injection.

17. The medicament of claim 15 which is applied topically.

18. The medicament of claim 15 wherein the medicament is used to attack a coronavirus, the SARS virus, the avian flu virus or a mutation of any one of the foregoing.

19. The medicament of claim 15 wherein said one or more sources comprises either a single source of both copper and silver ions or a plurality of sources, one of which is a source of copper ions and one of which is a source of silver ions, either of which may also serve a source of the other ion.

20. An improved cleansing composition wherein the improvement comprises the inclusion of an antivirally effective amount of one or more sources of silver ions and copper ions.

21. The improved antiviral cleansing composition of claim 20 wherein the weight ratio of copper to silver is from 20:1 to 1:20.

22. The improved antiviral cleaning composition of claim 20 wherein the amount of the one or more sources of silver ions and copper ions is from about 0.1 to about 30 percent by weight based on the total weight of the cleaning composition.

23. The improved cleaning composition of claim 20 wherein said one or more sources comprises either a single source of both copper and silver ions or a plurality of sources, one of which is a source of copper ions and one of which is a source of silver ions, either of which may also serve a source of the other ion.

24. An improved coating composition wherein the improvement comprises the presence of an antivirally effective amount of one or more sources of silver ions and copper ions.

25. The improved antiviral coating composition of claim 24 wherein the weight ratio of copper to silver is from 20:1 to 1:20.

26. The improved antiviral coating composition of claim 24 wherein the amount of the one or more sources of silver ions and copper ions is from about 1.0 to about 30 percent by weight based on the total weight of the coating composition.

27. The improved antiviral coating composition of claim 24 wherein said one or more sources comprises either a single source of both copper and silver ions or a plurality of sources, one of which is a source of copper ions and one of which is a source of silver ions, either of which may also serve a source of the other ion.

28. An improved molding composition wherein the improvement comprises the presence of an antivirally effective amount of one or more sources of silver ions and copper ions.

29. The improved antiviral molding composition of claim 28 wherein the weight ratio of copper to silver is from 20:1 to 1:20.

30. The improved antiviral molding composition of claim 28 wherein the amount of the one or more sources of silver ions and copper ions is from about 0.1 to about 30 percent by weight based on the total weight of the coating composition.

31. The improved antiviral molding composition of claim 28 wherein said one or more sources comprises either a single source of both copper and silver ions or a plurality of sources, one of which is a source of copper ions and one of which is a source of silver ions, either of which may also serve a source of the other ion.

32. The improved antiviral molding composition of claim 28 wherein the molding composition comprises a thermoplastic polymer.

33. The improved antiviral molding composition of claim 28 wherein the molding composition comprises a thermoset material.

34. The improved antiviral molding composition of claim 28 wherein the molding composition comprises a polymer film forming material.
